(19) United States
(12) Patent Application Publication
(10) Pub. No.: US 2010/0063009 A1
(43) Pub. Date: Mar. 11, 2010

(54) Pharmaceutical Combination Comprising 6-Dimethylaminomethyl-1-(3-methoxy-phenyl)-cyclohexane-1.3-diol and an NSAID

(75) Inventors: Petra Bloms-Funke, Wuerselen (DE); Klaus Schiene, Juechen (DE)

Correspondence Address: CROWELL & MORING LLP;INTELLECTUAL PROPERTY GROUP, P.O. BOX 14300, WASHINGTON, DC, 20044-4300, US

(73) Assignee: GRUENENTHAL GmbH, Aachen (DE)

(21) Appl. No.: 12/554,243

(22) Filed: Sep. 4, 2009

Related U.S. Application Data
(60) Provisional application No. 61/094,643, filed on Sep. 5, 2008.

(30) Foreign Application Priority Data
Sep. 5, 2008 (EP) 08 015 687.0

(51) Int. Cl. A61K 31/135; A61K 31/196; A61K 31/192; A61K 31/415; A61K 31/60; A61K 31/405; A61P 25/00; A61P 19/02

(52) U.S. Cl. 514/163; 514/646; 514/567; 514/570; 514/404; 514/569; 514/420

(57) ABSTRACT

A combination comprising (a) at least one 6-dimethylaminomethyl-1-(3-methoxy-phenyl)-cyclohexane-1,3-diol component, and (b) at least one non-steroidal anti-inflammatory drug (NSAID) component; a pharmaceutical composition and a dosage form comprising such as combination, and a method of treating pain and/or ostheoarthritis in a mammal characterized in that components (a) and (b) are administered simultaneously or sequentially to said mammal, in which component (a) may be administered before or after component (b), and in which components (a) or (b) are administered to the mammal either via the same or a different pathway of administration.

### CROSS REFERENCE TO RELATED APPLICATIONS

This application claims priority from co-pending U.S. provisional patent application No. 61/094,643, filed Sep. 5, 2008. Priority is also claimed based on European patent application no. EP 008 015 687.0, filed Sep. 5, 2008.

### BACKGROUND OF THE INVENTION

The present invention relates to a combination comprising (a) at least one 6-dimethylaminomethyl-1-(3-methoxy-phenyl)-cyclohexane-1,3-diol component, and (b) at least one non-steroidal anti-inflammatory drug (NSAID) component; a pharmaceutical combination and a dosage form comprising said combination as well as a method of treating one or more of pain and ostheoarthritis in a mammal characterized in that components (a) and (b) are administered simultaneously or sequentially to said mammal, wherein component (a) may be administered before or after component (b) and wherein components (a) or (b) are administered to the mammal either via the same or a different pathway of administration.

The treatment of pain conditions is extremely important in medicine. There is currently a worldwide demand for additional, not exclusively opioid-based, but highly effective pain treatment. The urgent need for action for patient-oriented and purposeful treatment of pain conditions, this being taken to mean the successful and satisfactory treatment of pain for the patient, is documented in the large number of scientific papers which have recently appeared in the field of applied analgesics and fundamental research work on nociception.

Even if the analgesics that are currently used for treating pain, for example opioids, NA- and 5HT-reuptake inhibitors, NSAIDS and COX inhibitors, are analgesically effective, side effects nevertheless sometimes occur. WO 2004/047823 describes substance combinations comprising certain analgesics including substituted 6-dimethylaminomethyl-1-phenyl-cyclohexane compounds and COX-II Inhibitors, which show super-additive effects upon administration. Due to the super-additive effect the overall dose and accordingly the risk of undesired side effects can be reduced.

### SUMMARY OF THE INVENTION

Thus, it was an object of the present invention to find further combinations that are suitable for the treatment of pain and which preferably exhibit fewer undesired side effects compared to its individual components, if administered in effective doses.

It has been found that a combination comprising (a) at least one 6-dimethylaminomethyl-1-(3-methoxy-phenyl)-cyclohexane-1,3-diol component, and (b) at least one non-steroidal anti-inflammatory drug (NSAID) component exhibits an analgesic effect. Said combination is also useful for the treatment of ostheoarthritis. If the components are present in the composition in such a weight ratio that a synergistic effect is observed after administration to the patient, the overall administered dose may be lowered, so that fewer undesired side-effects will occur.

Accordingly, the present invention relates to a combination comprising
(a) at least one 6-dimethylaminomethyl-1-(3-methoxy-phenyl)cyclohexane-1,3-diol component, and
(b) at least one non-steroidal anti-inflammatory drug component.

The 6-dimethylaminomethyl-1-(3-methoxy-phenyl)cyclohexane-1,3-diol component as used herein includes said compound in any possible form, thereby particularly including stereoisomers and salts. Also included are solvates and polymorphs of each of these forms. Included are also adducts, salts as well as reaction products of the 6-dimethylaminomethyl-1-(3-methoxy-phenyl)-cyclohexane-1,3-diol component and the NSAID component.

Thus, in one embodiment the present invention relates to a combination comprising
(a) 6-dimethylaminomethyl-1-(3-methoxy-phenyl)cyclohexane-1,3-diol of formula (I)

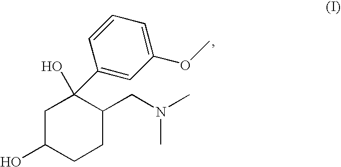

optionally in form of one of its stereoisomers, in particular an enantiomer or a diastereomer, a racemate or in form of a mixture of its stereoisomers, in particular enantiomers and/or diastereomers in any mixing ratio, or any corresponding salt thereof, and
(b) one or more non-steroidal anti-inflammatory drugs (NSAIDs).

In another embodiment the inventive combination comprises
(a) (1RS,3RS,6RS)-6-dimethylaminomethyl-1-(3-methoxy-phenyl)-cyclohexane-1,3-diol of formula (I′),

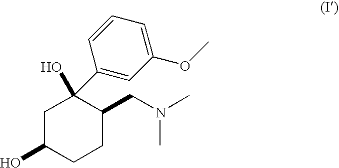

or a salt thereof, and
(b) one or more non-steroidal anti-inflammatory drugs (NSAIDs).

The (1RS,3RS,6RS)-6-dimethylaminomethyl-1-(3-methoxy-phenyl)-cyclohexane-1,3-diol stereoisomer of formula (I′) represents the racemate of the enantiomers (I′a) and (I′b):

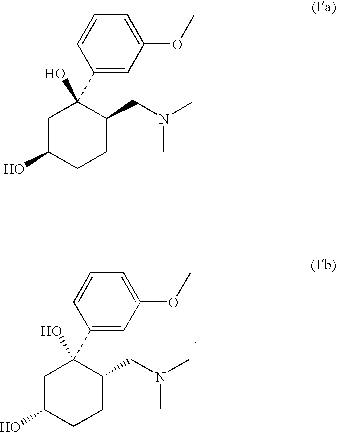

The compound 6-dimethylaminomethyl-1-(3-methoxy-phenyl)cyclohexane-1,3-diol of formula (I), its stereoisomers and corresponding salts thereof as well as methods for their preparation are well known, for example, from US RE 37,355 E, the entire disclosure of which is incorporated herein by reference. The compound (1RS,3RS,6RS)-6-dimethylaminomethyl-1-(3-methoxy-phenyl)-cyclohexane-1,3-diol is also known as Axomadol (WHO Drug Information, Vol. 17, No. 2, 2003, List 49).

If any of the components, particularly component (a), is present as mixture of enantiomers, such a mixture may contain the enantiomers in racemic or non-racemic form. A non-racemic form could, for example, could contain the enantiomers in a ratio of 60±5:40±5; 70±5:30±5; 80±5:20±5 or 90±5:10±5.

The term “isolated” as used herein in conjunction with a stereoisomer, in particular an enantiomer or a diastereomer, means that the stereoisomer, enantiomer or diastereomer is separated from its opposite stereoisomeric form, without necessarily being separated from other substances.

The compound 6-dimethylaminomethyl-1-(3-methoxy-phenyl)-cyclohexane-1,3-diol and its stereoisomers, in particular the (1RS,3RS,6RS)-stereoisomer, according to component (a) may be present in the inventive combination in form of a salt, preferably an acid addition salt, whereby any suitable acid capable of forming such an addition salt may be used.

The conversion of the 6-dimethylaminomethyl-1-(3-methoxy-phenyl)-cyclohexane-1,3-diol compound, particularly the (1RS,3RS,6RS)-stereoisomer, into a corresponding addition salt via reaction with a suitable acid may be effected in a manner well known to those skilled in the art. Suitable acids include, but are not limited to, hydrochloric acid, hydrobromic acid, sulfuric acid, methanesulfonic acid, formic acid, acetic acid, oxalic acid, succinic acid, tartaric acid, mandelic acid, fumaric acid, maleic acid, lactic acid, citric acid, glutamic acid, aspartic acid, 1,1-Dioxo-1,2-dihydro-1λ⁶-benzo[d]isothiazol-3-on (saccharin), monomethylsebacic acid, 5-oxo-proline, hexane-1-sulfonic acid, nicotinic acid, 2-, 3- or 4-amino benzoic acid, 2,4,6-trimethyl-benzoic acid, α-lipoic acid, acetyl glycine, and hippuric acid. Salt formation is preferably effected in a solvent, for example diethyl ether, diisopropyl ether, alkyl acetates, acetone and/or 2-butanone.

Certain salts of 6-dimethylaminomethyl-1-(3-methoxy-phenyl)-cyclohexane-1,3-diol, in particular of the (1RS,3RS,6RS)-stereoisomer, may be preferred such as the hydrochloride salt or the salts of phosphoric acid as well as polymorphs thereof. The salts of phosphoric acid and their respective polymorphs, are disclosed, for example, in US patent application publication no. 2006/0211887 A1, the entire disclosure of which is incorporated herein by reference.

Phosphoric acids that may be preferred are oxo acids of phosphorus. The di- (also pyro-) and the condensed meta- and polyphosphoric acids, which are also included according to the present invention can be derived from orthophosphoric acid (relative molar mass 98.0 g/mole).

Primary, secondary and tertiary phosphates, which are also included according to the present invention, can be formed by stepwise replacement of the H atoms of orthophosporic acid.

Phosphate salts as also included by the present invention are understood as meaning salts from the reaction of 6-dimethylaminomethyl-1-(3-methoxy-phenyl)-cyclohexane-1,3-diol of formula I in particular with condensed phosphoric acids, such as meta- and diphosporic acid, as well as salts of orthophosphoric acid.

Salts of diphosphoric acid and orthophosphoric acid are preferred. Salts of orthophosphoric acid are particularly preferred.

It is known to those skilled in the art that the analgesic action of NSAIDs is due to the inhibition of the enzymatic production of prostaglandins, wherein cyclooxygenase (COX) is the key enzyme in the conversion of arachidonic acid derived from lipids of the cell membrane to prostaglandins and other eicosanoids. COX exists in two different isoforms characterized by different expression patterns. COX-I is constitutively expressed in many cells of the body and responsible mainly for the production of eicosanoids serving normal physiological functions. COX-II expression is induced during inflammation and also COX-II is expressed in the central nervous system.

The term non-steroidal anti-inflammatory drug as used herein designates compounds showing essentially COX-I specific inhibition selective COX-I or mixed COX-I/II inhibition, so that selective COX-II Inhibitors are not encompassed. The term non-steroidal anti-inflammatory drugs of component (b) as used herein includes any possible form of these NSAIDs, particularly including stereoisomers such as enantiomers, solvates, salts and corresponding solvates and polymorphs thereof. For example, the term Ibuprofen as used herein particularly includes its racemic mixtures, its non-racemic mixtures, and its pure stereoisomer such as (S)-(+)-Ibuprofen, the term diclofenac as used herein may particularly include its salt diclofenac-sodium and the term metamizol may particularly include its salt metamizol-sodium.

Non-steroidal anti-inflammatory drugs as well as processes for their preparation are well known in the art, for example from E. Friderichs et al. “Analgesics and Antipyretics”, Ullmann's Encyclopedia of Industrial Chemistry, Sixth Edition, Wiley-VCH Verlag GmbH, Germany 2000, pages 1-22 and H. Buschmann, T. Christoph, E. Friderichs, C. Maul, B. Sundermann, “Analgesics—From Chemistry and Pharmacology to Clinical Application”, 2002, Part II, Wiley-VCH Verlag, Germany.

In one embodiment of the inventive combination component (b) is selected from the group consisting of acemetacin, acetylsalicylic acid, bufexamac, diclofenac, diclofenac-sodium, diflunisal, dipyrone (metamizol), metamizol-sodium, ethenzamide, etofenamate, flufenamic acid, flurbiprofen, ibuprofen, (+)-ibuprofen, (−)-ibuprofen, indomethacin, isoxicam, kebuzone, ketoprofen, ketorolac, lonazolac, lornoxicam, meclofenamic acid, mefenamic acid, mofebutazone, nabumetone, naproxen, (+)-naproxen, niflumic acid, oxaprozine, oxyphenbutazone, phenylbutazone, piroxicam, propyphenazone, salicylamide, sulindac, tenoxicam, tiaprofenic acid, SC560; sulfasalazine and tolmetin.

In another embodiment of the inventive combination component (b) is selected from the group consisting of acetylsalicylic acid, diclofenac, diclofenac-sodium, dipyrone (metamizol), metamizol-sodium, ibuprofen, (+)-ibuprofen, (−)-ibuprofen, indomethacin, naproxen and (+)-naproxen.

In yet another embodiment of the inventive combination component (b) is selected from the group consisting of diclofenac, diclofenac-sodium, dipyrone (metamizol), metamizol-sodium, ibuprofen, (+)-ibuprofen, (−)-ibuprofen, naproxen and (+)-naproxen.

Other specific embodiments of the combination according to the present invention comprise axomadol or a salt thereof and one or more NSAIDs selected from the group consisting of acetylsalicylic acid, diclofenac, diclofenac-sodium, dipyrone (metamizol), metamizol-sodium, ibuprofen, (+)-ibuprofen, (−)-ibuprofen, indomethacin, naproxen and (+)-naproxen.

Other specific embodiments of the combination according to the present invention comprise axomadol or a salt thereof and one or more NSAIDs selected from the group consisting of diclofenac, diclofenac-sodium, dipyrone (metamizol), metamizol-sodium, ibuprofen, (+)-ibuprofen, (−)-ibuprofen, naproxen and (+)-naproxen.

Both components (a) and (b) may be administered in their usual daily dosage. Preferably, the daily amount of diclofenac administered to a patient may be 25 to 300 mg, particularly preferably the amount may be 35 to 200 mg, yet more preferably 50 to 150 mg. Preferably the daily amount of ibuprofen administered to a patient may be 300 to 2400 mg, particularly preferably the amount may be 350 to 1600 mg, yet more preferably 400 to 1200 mg. Preferably, the daily amount of naproxen administered to a patient may be 1 to 1500 mg, preferably 5 to 1250 mg. Preferably, the daily amount of metamizol administered to a patient may be 1 to 4500 mg, preferably 5 to 4000 mg. The 6-dimethylaminomethyl-1-(3-methoxy-phenyl)cyclohexane-1,3-diol component, in particular the (1RS,3RS,6RS)-stereoisomer, may preferably be administered to a patient in a daily dosage of 1 to 1200 mg, particularly preferably in a dosage of 5 to 900 mg.

In another embodiment of the present invention the inventive combination may contain components (a) and (b) essentially in an equieffective ratio.

In yet a further embodiment of the inventive combination components (a) and (b) are present in such a weight ratio that the resulting composition will exert a synergistic effect upon administration to a patient. Suitable weight ratios can be determined by methods known to persons skilled in the art.

Both components (a) and (b) may also be present in the inventive combination in ratios deviating from the equieffective ratio. For, example, each of the components could be present in a range from 1/50 of the equieffective amount to 50 times the equieffective amount, from 1/20 of the equieffective amount to 20 times the equieffective amount, from 1/10 of the equieffective amount to 10 times the equieffective amount, from ⅕ of the equieffective amount to 5 times the equieffective amount, from ¼ of the equieffective amount to 4 times the equieffective amount, from ⅓ of the equieffective amount to 3 times the equieffective amount, or from ½ of the equieffective amount to 2 times the equieffective amount.

In another embodiment of the present invention the components (a) and (b) can be administered in a specific dosage regimen to treat one or more disorders selected from the group consisting of ostheoarthritis and pain, e.g. inflammatory pain, neuropathic pain, acute pain, chronic pain, visceral pain, migraine pain or cancer pain. Components (a) and (b) may be administered simultaneously or sequentially to one another, in each case via the same or different administration pathways. Another aspect of the present invention is therefore a method of treating one or more of ostheoarthritis and pain, e.g. inflammatory pain, neuropathic pain, acute pain, chronic pain, visceral pain, migraine pain or cancer pain, characterized in that components (a) and (b) are administered simultaneously or sequentially to a mammal, wherein component (a) may be administered before or after component (b) and wherein components (a) or (b) are administered to the mammal either via the same or a different pathway of administration. Suitable pathways of administration include, but are not limited to, oral, intravenous, intraperitoneal, transdermal, intrathecal, intramuscular, intranasal, transmucosal, subcutaneous, or rectal administration. A suitable embodiment would thus be a kit in which the components of the inventive combination, although spatially separated, are provided in a common presentation form.

Some non-steroidal anti-inflammatory drugs have a group that is capable of forming a salt with component (a). Such groups can be, for example, acidic groups such as carboxy groups. Suitable representatives include, for example, acetylsalicylic acid, indomethacin, ketoprofen, flurbiprofen, diclofenac and ibuprofen which may be used as such to form acid addition salts with the compound 6-dimethylaminomethyl-1-(3-methoxy-phenyl)-cyclohexane-1,3-diol of formula (I), thereby incorporating both components (a) and (b) in one and the same salt.

Thus, in another embodiment of the present invention the inventive combination comprises components (a) and (b) in form of a salt formed from these two components. Such a salt formation may be partial, i.e. the inventive composition comprises one or both of these components also in their non-salt form, or the salt formation may be essentially complete.

Accordingly, in another aspect the present invention relates to a salt formed from
(a) at least one 6-dimethylaminomethyl-1-(3-methoxy-phenyl)cyclohexane-1,3-diol of formula (I)

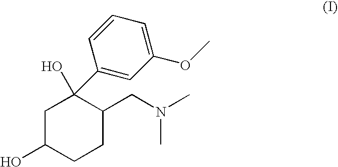

optionally in form of one of its stereoisomers, in particular an enantiomer or a diastereomer, a racemate or in form of a mixture of its stereoisomers, in particular enantiomers and/or diastereomers in any mixing ratio, and
(b) at least one non-steroidal anti-inflammatory drug (NSAID).

In another embodiment the present invention relates to a salt, wherein the cationic salt component is formed from (a) the compound 6-dimethylaminomethyl-1-(3-methoxy-phenyl)-cyclohexane-1,3-diol of formula (I) and the anionic salt component is formed from (b) an acidic non-steroidal anti-inflammatory drug.

In a further embodiment of the inventive salt component (a) is (1RS,3RS,6RS)-6-dimethylaminomethyl-1-(3-methoxy-phenyl)-cyclohexane-1,3-diol.

In yet another embodiment of the inventive salt the acidic non-steroidal anti-inflammatory drug is selected from acetylsalicylic acid, indomethacin, diclofenac, flurbiprofen, ibuprofen, (+)-ibuprofen, (−)-ibuprofen, ketoprofen, naproxen and (+)-naproxen.

The 6-dimethylaminomethyl-1-(3-methoxy-phenyl)cyclohexane-1,3-diol compound of component (a) and the NSAID component (b) may also be linked to one another, for example, via a covalent linkage. Such a covalent linkage may, for example, be obtained from the phenolic hydroxy group of component (a) and a carboxy group of an NSAID according to component (b), whereby an ester linkage is obtained. Such compounds combining both components (a) and (b) are also included by the present invention.

The inventive combinations, the inventive salts as well as the inventive compounds are toxicologically safe and are therefore suitable for the treatment of mammals, particularly humans including infants, children and grown-ups.

Thus, in a further aspect the present invention relates to a pharmaceutical composition comprising an inventive combination as described herein and/or a salt as described herein and/or a compound as described herein and optionally one or more auxiliary agents.

In a further aspect the present invention relates to a pharmaceutical dosage form comprising an inventive combination as described herein and/or a salt as described herein and/or a compound as described herein and one or more auxiliary agents. In one embodiment the inventive pharmaceutical dosage form additionally comprises caffeine.

In another embodiment, the inventive pharmaceutical dosage form is suitable for oral, intravenous, intraperitoneal, transdermal, intrathecal, intramuscular, intranasal, transmucosal, subcutaneous, or rectal administration.

The formulations and dosage forms of the invention may contain auxiliary agents, for example, carriers, fillers, solvents, diluents, colorants and/or binders. The selection of auxiliary agents and of the amounts of the same to be used depends, for example, on how the drug is to be administered.

Suitable auxiliary agents in the context of this invention may be any substances known to persons skilled in the art to be useful for the preparation of galenic formulations. Examples of suitable auxiliary agents include, but are not limited to, water, ethanol, 2-propanol, glycerol, ethylene glycol, propylene glycol, polyethylene glycol, polypropylene glycol, glucose, fructose, lactose, saccharose, dextrose, molasses, starch, modified starch, gelatine, sorbitol, inositol, mannitol, microcrystalline cellulose, methyl cellulose, carboxymethyl cellulose, cellulose acetate, shellac, cetyl alcohol, polyvinyl pyrrolidone, paraffins, waxes, natural and synthetic gums, acacia gum, alginates, dextran, saturated and unsaturated fatty acids, stearic acid, magnesium stearate, zinc stearate, glycerol stearate, sodium lauryl sulfate, edible oils, sesame oil, coconut oil, peanut oil, soybean oil, lecithin, sodium lactate, polyoxyethylene and polypropylene fatty acid ester, sorbitan fatty acid ester, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulfate, zinc sulfate, calcium sulfate, potash, calcium phosphate, dicalcium phosphate, potassium bromide, potassium iodide, talcum, kaolin, pectin, crosspovidone, agar and bentonite.

Pharmaceutical formulations (dosage forms) in the form of tablets, effervescent tablets, chewing tablets, dragees, capsules, drops, juices or syrups are, for example, suitable for oral administration. Oral pharmaceutical formulations may also be in the form of multiparticulates such as granules, pellets, spheres, crystals and the like, optionally compressed into a tablet, filled into a capsule, filled into a sachet or suspended in a suitable liquid medium. Suitable oral pharmaceutical formulations may also be equipped with an enteric coating.

Pharmaceutical formulations that are suitable for parenteral, topical and inhalative administration include, but are not limited to, solutions, suspensions, easily reconstitutable dry preparations and sprays. Suppositories are a suitable pharmaceutical formulation for rectal administration. Examples of suitable formulations for percutaneous administration include formulations in a deposit, in dissolved form, for example, in a patch optionally with the addition of agents to promote skin penetration.

One or both of the components (a) and (b) and/or the inventive salt and/or the inventive compound may be present in the inventive pharmaceutical combination/formulation at least partially in controlled-release form. Moreover, any controlled release/immediate release combination of said components may also be present in the inventive pharmaceutical formulation. For example, one or both of the components may be released from the inventive formulation with a certain delay, e.g. if administered orally or rectally. Such formulations are particularly useful for “once-daily” or “twice-daily” preparations, which only have to be taken once a day, or twice a day, respectively. Suitable controlled-release materials are known to persons skilled in the art, e.g. from US patent publication no. 2006/121113 A1, which is hereby incorporated by reference in its entirety.

The pharmaceutical formulations of the invention may be produced using materials, means, devices and processes that are well known in the art of pharmaceutical formulations, as described for example in “Remington's Pharmaceutical Sciences”, A. R. Gennaro (ed.), 17^{th} edition, Mack Publishing Company, Easton, Pa. (1985), in particular in part 8, chapters 76 to 93.

In order to obtain a solid pharmaceutical formulation such as a tablet, pill or capsule for example, the components of the pharmaceutical composition may be granulated with a pharmaceutical carrier, for example conventional tablet ingredients such as corn starch, lactose, saccharose, sorbitol, talcum, magnesium stearate, dicalcium phosphate or pharmaceutically acceptable gums, and pharmaceutical diluents, for example water, in order to form a solid composition that contains the components in homogeneous distribution. The term “homogeneous distribution” is taken to mean that the components are distributed uniformly over the entire composition, so that said composition may easily be divided into equally effective unit dose forms, such as tablets, pills or capsules. The solid composition is then divided into unit dose forms. The tablets or pills of the pharmaceutical composition according to the invention may also be coated or compounded in a different manner, in order to provide a dose form with a controlled release.

The amount of the inventive pharmaceutically active combination, salt, or compound to be administered to the patient may vary depending on different factors known to persons skilled in the art, for example, the weight of the patient, the route of administration, the severity of the illness and the like.

In a further aspect the present invention relates to the use of an inventive combination as described herein and/or a pharmaceutical salt as described herein and/or a compound as described herein for the treatment of one or more disorders selected from the group consisting ostheoarthritis and pain.

In another aspect the present invention relates to the use of an inventive combination as described herein and/or a pharmaceutical salt as described herein and/or a compound as described herein for the preparation of a medicament for the treatment of one or more disorders selected from the group consisting of ostheoarthritis and pain.

In yet another aspect the present invention relates to a method of treating one or more of ostheoarthritis and pain in a mammal, preferably a human, which comprises administering an effective amount of an inventive combination as described herein and/or a pharmaceutical salt as described herein and/or a compound as described herein to the mammal.

The term pain as used herein includes, but is not limited to, inflammatory pain, neuropathic pain, acute pain, chronic pain, visceral pain, migraine pain and cancer pain.

### Pharmacological Test Methods

### A. Randall-Selitto Test in Rats

The weight ratios of the components (a) and (b) that will lead to a supra-additive effect (synergistic effect) of the inventive pharmaceutical composition may be determined via the test of Randall and Selitto as described in Arch. Int. Pharmacodyn., 1957, 111: 409 to 419, which is a model for inflammatory pain. The respective part of the literature is hereby incorporated by reference and forms part of the present disclosure.

Acute inflammation is induced by an intraplantar injection of 0.1 ml of a carrageenan solution (0.5% in distilled water) into one hind paw. The mechanical nociceptive threshold is measured 4 hours after carrageenan injection using an Algesiometer (Ugo Basile, Italy). The device generates a mechanical force with a linear increase over time. The force is applied to the dorsal surface of the inflamed rat hind paw via a cone-shaped stylus with a rounded tip (2 mm tip diameter). The nociceptive threshold is defined as the force (in grams) at which the rat vocalises (cut-off force 250 g). The mechanical nociceptive threshold is measured at different timepoints after the drug or vehicle administration. The antinociceptive and antihyperalgesic activity of the tested substance is expressed as percentages of the maximal possible effect (% MPE). The group size is n=10.

The analysis of the results with respect to a supra-additive effect of the inventive pharmaceutical composition comprising the components (a) and (b) is carried out via statistical comparison of the theoretical additive ED₅₀-value with the experimentally determined ED₅₀-value of a so-called fixed ratio combination (isobolographic analysis according to Tallarida J T, Porreca F, and Cowan A. Statistical analysis of drug-drug and site-site interactions with isobolograms. Life Sci 1989; 45: 947-961).

The interactions studies presented herein were performed using equieffective doses of the two components, calculated from the ratio of the respective ED₅₀ values of the components if administered alone.

The route of administration was intravenous (i.v.) for (1RS,3RS,6RS)-6-dimethylaminomethyl-1-(3-methoxy-phenyl)-cyclohexane-1,3-diol hydrochloride (A) and intraperitoneal (i.p.) for the NSAIDs diclofenac, ibuprofen, metamizol-sodium and naproxen. When A was applied alone, the peak effect was reached 15 min p. appl. (timepoint of first measurement) and ED₅₀-value of 15.80 (14.46-17.36) mg/kg i.v., 14.5 (13.3-15.6) mg/kg i.v. and 14.1 (13.2-15.1) mg/kg i.v. were calculated. The NSAIDs induced dose-dependent analgesic effects with ED₅₀-values of 145.9 (134.8-155.1) mg/kg i.p. (diclofenac), 138.8 (130.3-147.1) mg/kg i.p. (Ibuprofen), 88.1 (77.5-98.3) mg/kg i.p. (metamizol-sodium) and 164 (158-169) mg/kg i.p. (naproxen), reaching the peak effect 30 min p. appl. (diclofenac, ibuprofen) and 45 min p. appl. (naproxen, metamizol-sodium). According to their respective timepoint of peak effect, A was applied 15 min and the NSAID component 30 min or 45 min before timepoint of measurement of the interaction-experiments (i. e. the NSAID component was applied 15 min (diclofenac, ibuprofen) and 30 min (naproxen, metamizol-sodium) before A. Thus, the time point of ED₅₀ calculation of the combination corresponds to the timepoint of the peak effect of the respective compound. The isobolographic analysis revealed that the experimental ED₅₀-values of the combinations were significantly lower than the respective theoretical ED₅₀-values. Thus, the combination studies demonstrate significant synergistic interaction of A with all of the NSAIDs, Diclofenac, Ibuprofen, Metamizol-Sodium and Naproxen.

The results of the isobolographic analysis are summarized in the following Table 1:

TABLE 1
Experimental ED₅₀ values of A, Diclofenac, Ibuprofen, Metamizol-Sodium and Naproxen and isobolographic analysis of
the interaction between A with these NSAIDs, respectively.

| Substance/ | | | | | | Theoretical | Experimental | |
|---|---|---|---|---|---|---|---|---|
| ED₅₀ | | | | Metamizol- | | ED₅₀ of the | ED₅₀ of | |
| [mg/kg] | A | Ibuprofen | Diclofenac | Sodium | Naproxen | combination | combination | Interaction |
| A + | 15.80 | 138.8 | — | — | — | 77.31 | 54.23 | supra- |
| Ibuprofen | (14.46-17.36)* | (130.3-147.1) | | | | (73.12-81.49) | (48.55-60.75) | additive |
| | | | | | | | | (p < 0.001) |
| A + | 15.80 | — | 145.9 | — | — | 80.83 | 53.67 | supra- |
| Diclofenac | (14.46-17.36)* | | (134.8-155.1) | | | (76.23-85.42) | (49.99-58.09) | additive |
| | | | | | | | | (p < 0.001) |
| A + | 14.5 | — | — | 88.1 | — | 51.3 | 38.2 | supra- |
| Metamizol- | (13.3-15.6) | | | (77.5-98.3) | | (47.7-54.9) | (33.1-42.3) | additive |
| Sodium | | | | | | | | (p < 0.001) |
| A + | 14.1 | — | — | — | 164 | 89.2 | 37.3 | supra- |
| Naproxen | (13.2-15.1) | | | | (158-169) | (79.1-85.1) | (32.3-41.3) | additive |
| | | | | | | | | (p < 0.001) |

*identical single-substance group with A for these combinations
p: level of statistical significance

The following examples are intended to illustrate the invention without restricting the scope of the invention to these examples.

### EXAMPLES

### Example 1

(1RS,3RS,6RS)-6-dimethylaminomethyl-1-(3-methoxy-phenyl)-cyclohexane-1,3-diol (100 mg, 0.358 mmol) was dissolved in ethanol under heating. Acetylsalicylic acid (64.5 mg, 0.358 mmol) was dissolved in water under heating. Both solutions were combined and heated under reflux over night. Subsequently the reaction mixture was concentrated in vacuo.

Yield: 200 mg (>99%) (pink oil)

Melting point: 127.4° C.

### Example 2

A solution of (1RS,3RS,6RS)-6-dimethylaminomethyl-1-(3-methoxy-phenyl)-cyclohexane-1,3-diol (100 mg, 0.358 mmol) and Ibuprofen (74 mg, 0.358 mmol) in acetone was stirred at 30-40° C. overnight. The reaction mixture was cooled to 5-10° C. upon which slight crystallization was observed.

Melting point: 89.5° C.

### Example 3

A solution of (1RS,3RS,6RS)-6-dimethylaminomethyl-1-(3-methoxy-phenyl)-cyclohexane-1,3-diol (100 mg, 0.358 mmol) and (S)(+)-Ibuprofen (74 mg, 0.358 mmol) in acetone (300 μl) was stirred at 45° C. over night and then kept at room temperature for 3 days. The reaction mixture was then cooled to 5-10° C. upon which slight crystallization was observed. The reaction mixture was concentrated in vacuo and cooled to approximately −20° C. to facilitate crystallization.

Yield: 80 mg (46%)

Melting point: 98.1° C.

### Example 4

A solution of (1RS,3RS,6RS)-6-dimethylaminomethyl-1-(3-methoxy-phenyl)-cyclohexane-1,3-diol (100 mg, 0.358 mmol) and Naproxen (0.358 mmol) in acetone (300 μl) was stirred at 45° C. over night and then kept at room temperature for 3 days. The reaction mixture was cooled to 5-10° C. upon which slight crystallization was observed. Subsequently the reaction mixture was concentrated in vacuo.

Yield: 173 mg (94%) (white solid)

### Example 5

A solution of (1RS,3RS,6RS)-6-dimethylaminomethyl-1-(3-methoxy-phenyl)-cyclohexane-1,3-diol (100 mg, 0.358 mmol) and Indomethacin (128 mg, 0.358 mmol) in acetone (300 μl) was stirred at 45° C. over night and then kept at room temperature for 3 days. The reaction mixture was cooled to 5-10° C. upon which slight crystallisation was observed. The reaction mixture was concentrated in vacuo and cooled to approximately −20° C. to facilitate crystallization.

Yield: 120 mg (53%) (crystalline)

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A composition of matter comprising in combination:
(a) a 6-dimethylaminomethyl-1-(3-methoxy-phenyl)cyclohexane-1,3-diol compound, and
(b) a non-steroidal anti-inflammatory drug.

2. A composition of matter as claimed in claim 1, wherein the 6-dimethylaminomethyl-1-(3-methoxy-phenyl)-cyclohexane-1,3-diol compound corresponds to formula I: 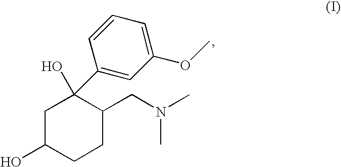
or a pharmaceutically acceptable salt thereof.

3. A composition of matter as claimed in claim 1, wherein said 6-dimethylaminomethyl-1-(3-methoxy-phenyl)cyclohexane-1,3-diol compound is in the form of a mixture of stereoisomers in any mixing ratio.

4. A composition of matter as claimed in claim 3, wherein said mixture is a racemic mixture.

5. A composition of matter as claimed in claim 1, wherein said 6-dimethylaminomethyl-1-(3-methoxy-phenyl)cyclohexane-1,3-diol compound is in the form of an isolated stereoisomer.

6. A composition of matter as claimed in claim 1, wherein said 6-dimethylaminomethyl-1-(3-methoxy-phenyl)cyclohexane-1,3-diol compound is (1RS,3RS,6RS)-6-dimethylaminomethyl-1-(3-methoxy-phenyl)-cyclohexane-1,3-diol or a pharmaceutically acceptable salt thereof.

7. A composition of matter as claimed in claim 6, wherein said 6-dimethylaminomethyl-1-(3-methoxy-phenyl)cyclohexane-1,3-diol compound is a hydrochloride salt or phosphoric acid salt of (1RS,3RS,6RS)-6-dimethylaminomethyl-1-(3-methoxy-phenyl)-cyclohexane-1,3-diol.

8. A composition of matter as claimed in claim 1, wherein said non-steroidal anti-inflammatory drug is selected from the group consisting of acemetacin, acetylsalicylic acid, bufexamac, diclofenac, diclofenac-sodium, diflunisal, aipyrone, metamizol-sodium, ethenzamide, etofenamate, flufenamic acid, flurbiprofen, ibuprofen, (+)-ibuprofen, (−)-ibuprofen, indomethacin, isoxicam, kebuzone, ketoprofen, ketorolac, lonazolac, lornoxicam, meclofenamic acid, mefenamic acid, mofebutazone, nabumetone, naproxen, (+)-naproxen, niflumic acid, oxaprozine, oxyphenbutazone, phenylbutazone, piroxicam, propyphenazone, salicylamide, sulindac, tenoxicam, tiaprofenic acid, SC560, sulfasalazine and tolmetin.

9. A composition of matter as claimed in claim 8, wherein said non-steroidal anti-inflammatory drug is selected from the group consisting of acetylsalicylic acid, diclofenac, diclofenac-sodium, dipyrone, metamizol-sodium, ibuprofen, (+)-ibuprofen, (−)-ibuprofen, indomethacin, naproxen and (+)-naproxen.

10. A composition of matter as claimed in claim 1, wherein said 6-dimethylaminomethyl-1-(3-methoxy-phenyl)cyclohexane-1,3-diol compound and said non-steroidal anti-inflammatory drug are at least partially present as an adduct, a salt or a compound formed from 6-dimethylaminomethyl-1-(3-methoxy-phenyl)-cyclohexane-1,3-diol compound and said non-steroidal anti-inflammatory drug.

11. A composition of matter as claimed in claim 1, wherein said 6-dimethylaminomethyl-1-(3-methoxy-phenyl)cyclohexane-1,3-diol compound and said non-steroidal anti-inflammatory drug are present in a weight ratio such that the composition exerts a synergistic effect upon administration to a patient.

12. A pharmaceutical dosage form comprising a composition of matter as claimed in claim 1, formulated for oral, intravenous, intraperitoneal, intradermal, intrathecal, intramuscular, intranasal, transmucosal, subcutaneous, or rectal administration to a patient.

13. A pharmaceutical dosage form as claimed in claim 12, wherein at least one of said 6-dimethylaminomethyl-1-(3-methoxy-phenyl)cyclohexane-1,3-diol compound and said non-steroidal anti-inflammatory drug is present in controlled-release form.

14. A method of treating a condition selected from the group consisting of osteoarthritis and pain in a subject, said method comprising administering to said subject a pharmacologically effective amount of a composition of matter as claimed in claim 1.

15. A method as claimed in claim 14, wherein said condition is pain is selected from the group consisting of inflammatory pain, neuropathic pain, acute pain, chronic pain, visceral pain, migraine pain and cancer pain.

16. A method as claimed in claim 14, wherein said subject is a mammal.

17. A method as claimed in claim 14, wherein said 6-dimethylaminomethyl-1-(3-methoxy-phenyl)-cyclohexane-1,3-diol compound and said non-steroidal anti-inflammatory drug are administered simultaneously either by the same or by a different pathway of administration.

18. A method as claimed in claim 14, wherein said 6-dimethylaminomethyl-1-(3-methoxy-phenyl)-cyclohexane-1,3-diol compound and said non-steroidal anti-inflammatory drug are administered sequentially with the 6-dimethylaminomethyl-1-(3-methoxy-phenyl)-cyclohexane-1,3-diol compound being administered before the non-steroidal anti-inflammatory drug either by the same or by a different pathway of administration.

19. A method as claimed in claim 18, wherein said 6-dimethylaminomethyl-1-(3-methoxy-phenyl)-cyclohexane-1,3-diol compound is administered by a different pathway of administration from said non-steroidal anti-inflammatory drug.

20. A method as claimed in claim 14, wherein said 6-dimethylaminomethyl-1-(3-methoxy-phenyl)-cyclohexane-1,3-diol compound and said non-steroidal anti-inflammatory drug are administered sequentially with the 6-dimethylaminomethyl-1-(3-methoxy-phenyl)-cyclohexane-1,3-diol compound being administered after the non-steroidal anti-inflammatory drug either by the same or by a different pathway of administration.

21. A method as claimed in claim 20, wherein said 6-dimethylaminomethyl-1-(3-methoxy-phenyl)-cyclohexane-1,3-diol compound is administered by a different pathway of administration from said non-steroidal anti-inflammatory drug.
